(19) United States
(12) Patent Application Publication
(10) Pub. No.: US 2026/0210403 A1
(43) Pub. Date: Jul. 23, 2026

(54) CROSS ROLLER BEARING

(71) Applicant: BEARING ART Corp., Gyeongju-si (KR)

(72) Inventors: KEUN PYO LEE, Seoul (KR); DU HAK HEO, Seoul (KR); SANG JUN LEE, Seoul (KR); WOON JU LEE, Seoul (KR)

(73) Assignee: BEARING ART Corp., Gyeongju-si (KR)

(21) Appl. No.: 19/440,668

(22) Filed: Jan. 6, 2026

(30) Foreign Application Priority Data
Jan. 17, 2025 (KR) 10-2025-0007568

(51) Int. Cl. F16C 33/78; F16C 33/58

(52) U.S. Cl. CPC F16C 33/782; F16C 33/585; F16C 33/7803

(57) ABSTRACT

A cross roller bearing includes: an outer ring having an outer ring raceway formed on an inner circumferential side thereof; an inner ring located inside the outer ring and having an inner ring raceway formed on an outer circumferential side thereof to face the outer ring raceway; a plurality of rolling elements disposed between the outer ring raceway and the inner ring raceway, and orthogonally arranged in a circumferential direction; and a seal installed in an opening between the outer ring and the inner ring.

### CROSS-REFERENCE TO RELATED APPLICATIONS

This application is based on and claims priority under 35 U.S.C. § 119 to Korean Patent Application No. 10-2025-0007568 filed on Jan. 17, 2025, in the Korean Intellectual Property Office, the disclosure of which is incorporated by reference herein in its entirety.

### BACKGROUND OF THE INVENTION

### Field of the Invention

The present invention relates to a cross roller bearing, and more specifically, to a cross roller bearing with improved seal assembly deformation.

### Background Art

As is known, a cross roller bearing includes an inner ring and an outer ring, a toroidal roller raceway having a square cross section formed between the inner ring and the outer ring, and a plurality of cylindrical rollers inserted into the roller raceway to be electrically powered in a state of being alternately orthogonal to each other in the circumferential direction.

In the cross roller bearing having the integral inner ring and the integral outer ring, a roller insertion groove or a roller insertion hole is formed in the inner ring or the outer ring to allow insertion of the cylindrical rollers into the roller raceway. After the cylindrical rollers are inserted, the roller insertion groove or the roller insertion hole is sealed with a stopper. FIG. 2 of Korean Patent Publication No. 10-2018-0085789 illustrates a configuration in which a roller insertion groove is formed in an inner ring and sealed with a stopper.

As illustrated in FIGS. 1 and 2, an outer ring 11 of a cross roller bearing 10 is provided with a seal insertion part 16 into which a seal 18 is press-fitted (in the direction indicated by arrow “A” in FIG. 2). An inner ring 13 is provided on the inner side of the outer ring 11. An outer ring raceway 12 is formed on the inner circumferential side of the outer ring 11, and an inner ring raceway 14 is formed on the outer circumferential side of the inner ring 13. The outer ring raceway 12 and the inner ring raceway 14 face each other to form a roller raceway, and a plurality of cylindrical rolling elements 15 are disposed between the outer raceway 12 and the inner ring raceway 14 to be alternately orthogonal in the circumferential direction. Although FIGS. 1 and 2 illustrate gaps between the rolling elements 15 and the outer and inner ring raceways 12 and 14, the illustration is to clarify the outer ring raceway 12 and the inner ring raceway 14.

The seal insertion part 16 includes an insertion step portion 16-1 that protrudes outward in the axial direction, and a cylindrical seal contact portion 16-2 that extends outward in the axial direction from the radial outer side of the insertion step portion 16-1.

The seal 18 has a bent cross-section and includes a seal support 18-1 having a support insertion portion 18-11 that extends inward in the axial direction from the radial outer side, and a seal body 18-2 coupled to the seal support 18-1. The seal support 18-1 is made of a metallic material, and the seal body 18-2 is made of an elastomeric material or the like.

The seal body 18-2 includes a body insertion portion 18-21 that protrudes radially outward beyond the support insertion portion 18-11 from the axial outer side, and at least one seal lip 18-22 that protrudes radially inward. The seal lip 18-22 is in contact with the outer circumferential surface of the inner ring 13 on the outer side of the inner ring raceway 14.

The support insertion portion 18-11 has a cylindrical shape, and the outer diameter of the support insertion portion 18-11 is larger than the inner diameter of the seal contact portion 16-2.

As the seal 18 is inserted into the seal insertion part 16, the outer ring 11 is deformed in the directions indicated by arrows “B” and “C” in FIG. 2, resulting in deformation of the outer ring raceway 12 as shown by the dotted line “C”, and the outer diameter of the outer ring 11 is deformed as shown by the dotted line “B”. In this instance, note that the dotted lines “B” and “C” are exaggerated to describe the deformation more clearly.

Such deformation of the outer ring 11 is caused by the external force exerted by the support insertion portion 18-11 pressing the seal contact portion 16-2 outward as the support insertion portion 18-11 is inserted into the seal contact portion 16-2, and the external force exerted by the body insertion portion 18-21 pressing the seal contact portion 16-2 outward from the axial outer side as the body insertion portion 18-21 is inserted into the seal contact portion 16-2. In particular, when the deformation amount of the body insertion portion 18-21 exceeds the elastic limit, the deformation of the outer ring 11 rapidly increases.

The deformation of the outer ring 11 causes problems such as reduced rigidity against moment loads, assembly failure, and grease leakage. For reference, the rigidity against moment loads is measured by installing the bearing in a shaft and a housing, fixing the housing, applying a moment load to the shaft, and measuring the angular displacement of the shaft.

Such problems occurring in the cross roller bearing 10 have become a cause of increased vibration when the cross roller bearing 10 is installed in a harmonic reducer and mounted on a robot or the like for driving.

### PATENT LITERATURE

### Patent Documents

Korean Patent Publication No. 10-2018-0085789

### SUMMARY OF THE INVENTION

Accordingly, the present invention has been made to solve the above-mentioned problems occurring in the prior arts, and it is an objective of the present invention to provide a cross roller bearing with improved deformation during seal assembly.

To accomplish the above object, according to the present invention, there is provided a cross roller bearing including: an outer ring having an outer ring raceway formed on an inner circumferential side thereof; an inner ring located inside the outer ring and having an inner ring raceway formed on an outer circumferential side thereof to face the outer ring raceway; a plurality of rolling elements disposed between the outer ring raceway and the inner ring raceway, and orthogonally arranged in a circumferential direction; and a seal installed in an opening between the outer ring and the inner ring, wherein the outer ring includes a seal insertion part into which the seal is inserted, and the seal insertion part includes an insertion step portion protruding outward in an axial direction and a seal contact portion extending outward in the axial direction from a radial outer side of the insertion step portion, and the seal insertion part further includes an insertion guide portion formed on an axial outer side of the seal contact portion and an outer contact portion extending in the axial direction.

Moreover, the seal contact portion and the outer contact portion are formed in a cylindrical shape, and an inner diameter of the outer contact portion is greater than an inner diameter of the seal contact portion.

Furthermore, the seal includes a seal support and a seal body coupled to the seal support. The seal support includes a support insertion portion that is bent and extends axially inward from a radial outer side, and the seal body includes a body insertion portion protruding radially beyond the support insertion portion on an axial outer side of the support insertion portion. The seal is inserted into the seal insertion part such that the support insertion portion comes into contact with the seal contact portion and the body insertion portion comes into contact with the outer contact portion.

Additionally, a seal recess is concavely formed between the support insertion portion and the body insertion portion on an outer circumferential side of the seal. A space is formed among the seal recess, the seal contact portion, a contact step portion, and the outer contact portion, and when the body insertion portion is deformed in contact with the outer contact portion, a portion of the body insertion portion moves into the space.

In addition, the body insertion portion includes inclined portions on both axial sides such that a width in a radial direction decreases outward, and an axial width of an outer surface of the body insertion portion is smaller than an axial width of the outer contact portion.

Moreover, the contact step portion is inclined outward in the axial direction and inward in the radial direction. A bottom surface of the space is formed by the seal body and inwardly spaced from the seal contact portion to face the seal contact portion, and a first inclined portion of the body insertion portion, which is disposed on an axial inner side of the body insertion portion, is spaced apart from the contact step portion to face the contact step portion.

Furthermore, when a radial distance measured from the seal contact portion to the outer surface of the body insertion portion is defined as D2, and a radial distance from the seal contact portion to the outer contact portion is defined as D1, a compression ratio α of the body insertion portion compressed by insertion into the outer contact portion is calculated as:
α=(D2−D1)/D2, wherein the compression ratio α is greater than 0 and less than 0.3.

Additionally, when an axial width of the outer contact portion is defined as L1, an axial width of the body insertion portion based on the seal contact portion is defined as L2, an axial width of the outer surface of the body insertion portion is defined as L3, and an inclination angle of the contact step portion with respect to the radial direction is defined as θ, a filling rate (β) of the seal body is calculated as:
β=[(L2×D2)−{(L2−L3)/2×D2}]/[(L1×D1)+(D1×D1/2 tanθ)], wherein the filling rate β is in a range of 0.6 to 0.9.

According to the present invention described above, the cross roller bearing can improve deformation during seal assembly since there is no change in stiffness against moment loads before and after the seal is assembled.

Additionally, the cross roller bearing according to the present invention significantly reduces lubricant leakage and greatly reduces torque ripple when installed in a harmonic reducer and operated, thereby significantly reducing vibration when mounted on a robot or the like.

### BRIEF DESCRIPTION OF THE DRAWINGS

FIG. 1 is a partial cross-sectional view of a conventional cross roller bearing.

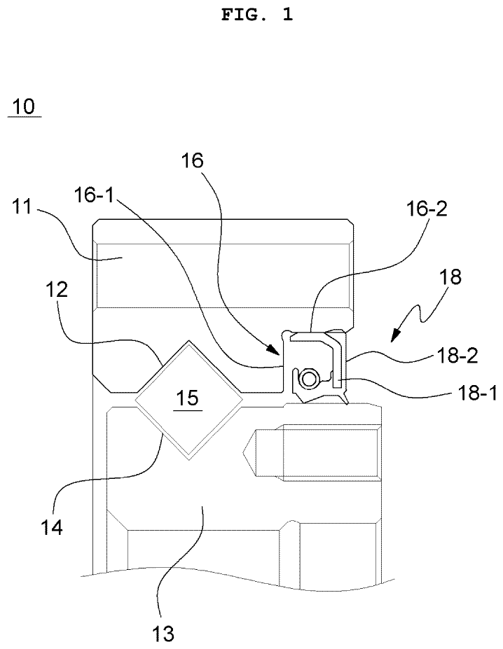

FIG. 2 is a diagram illustrating seal assembly of the conventional cross roller bearing.

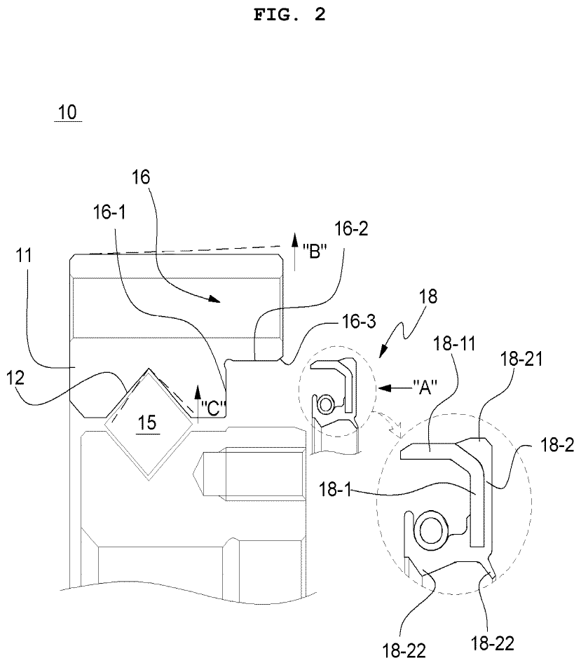

FIG. 3 is a partial cross-sectional view of a cross roller bearing according to the present invention.

FIG. 4 is a diagram illustrating seal assembly of the cross roller bearing according to the present invention.

FIG. 5 is an enlarged view of portion “A” of FIG. 3.

FIG. 6 is a graph illustrating torque variation according to rotational speed when the conventional cross roller bearing and the cross roller bearing according to the present invention are installed and operated in harmonic reducers, respectively.

FIG. 7 is a graph illustrating stiffness variation with respect to moment before and after seal insertion in the conventional cross roller bearing and the cross roller bearing according to the present invention.

### DETAILED DESCRIPTION OF THE PREFERRED EMBODIMENT

All technical and scientific terms used in the present specification, unless otherwise defined, have the same meaning as commonly understood by those of ordinary skill in the art to which the present invention belongs. The terms used herein are selected to best describe the present invention and are not intended to limit the scope of the present invention.

The expressions such as “include,” “comprise,” or “have” used in the present specification should be interpreted as open-ended terms that allow the inclusion of other components, unless explicitly stated otherwise.

The singular forms used in the present invention may also include plural forms unless the context clearly indicates otherwise. The above applies equally to the expressions used in the claims.

The terms “first,” “second,” and the like used in the specification are intended only to distinguish between multiple elements, and do not imply any particular order or importance.

When a component is described as being “connected to” or “coupled to” another component, it is to be understood that the component may be directly connected or coupled to the other component or may be indirectly connected or coupled through another component.

Hereinafter, a cross roller bearing according to the present invention will be described in detail with reference to the accompanying drawings.

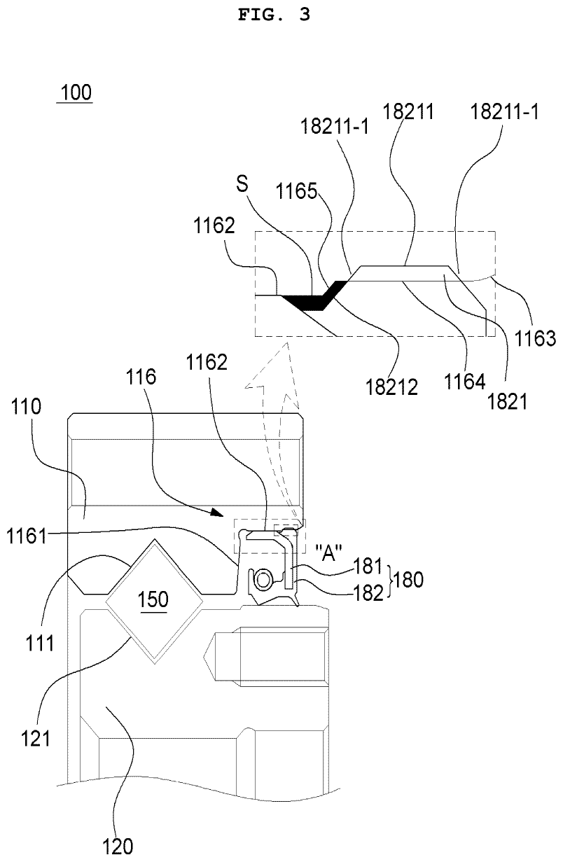

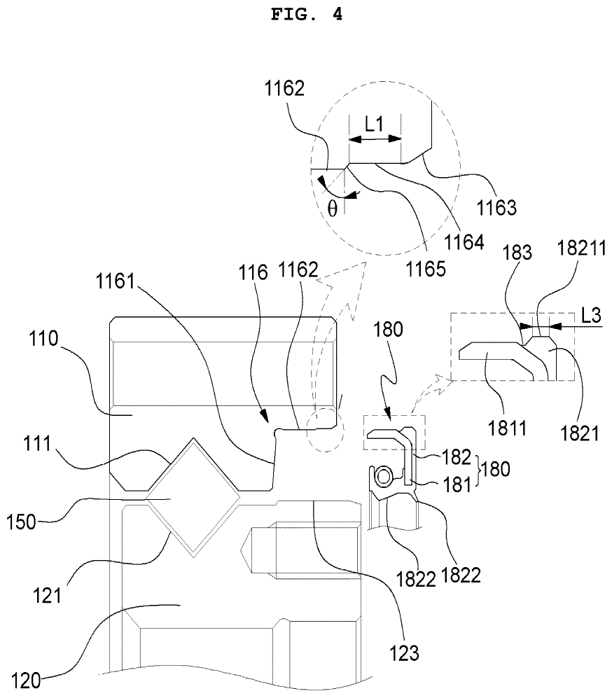

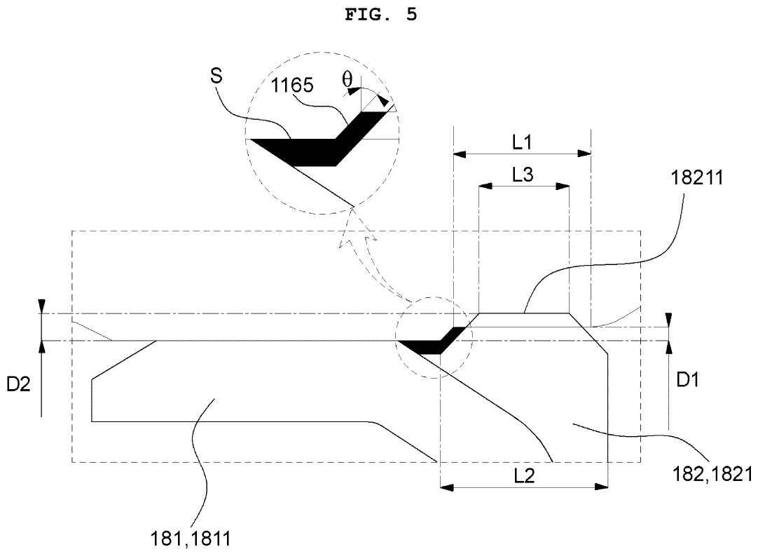

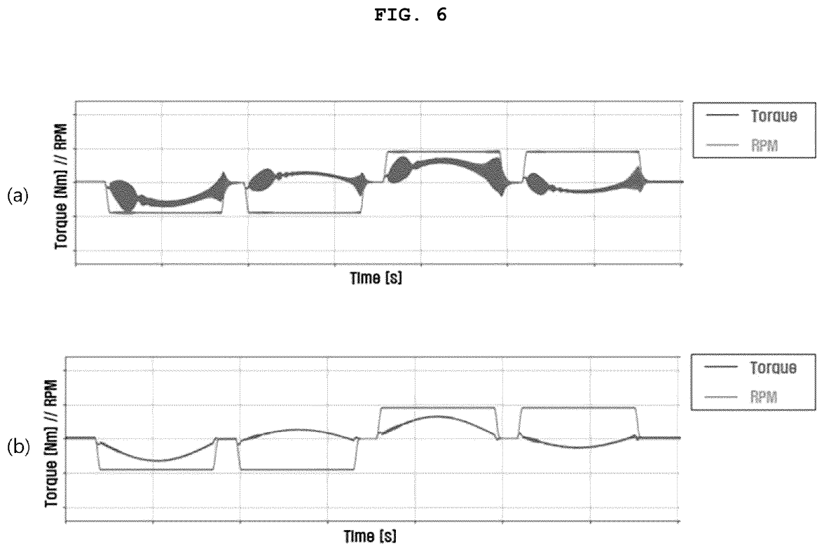

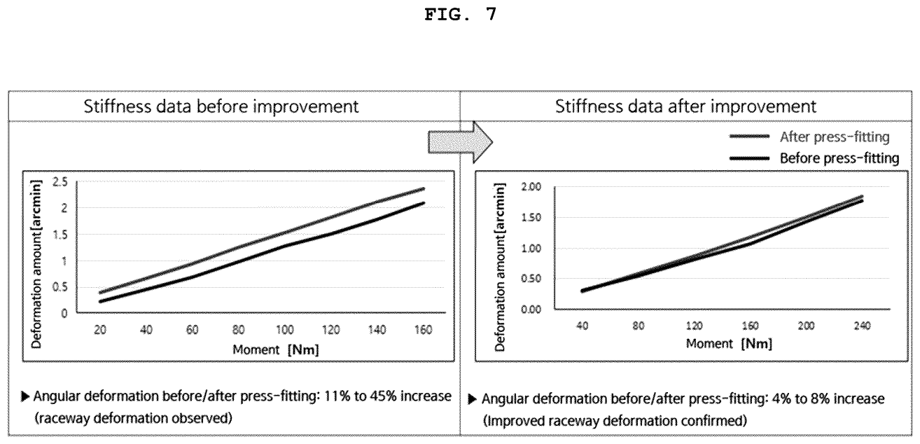

FIG. 3 is a partial cross-sectional view of a cross roller bearing according to the present invention, FIG. 4 is a diagram illustrating seal assembly of the cross roller bearing according to the present invention, FIG. 5 is an enlarged view of portion “A” of FIG. 3, FIG. 6 is a graph illustrating torque variation according to rotational speed when the conventional cross roller bearing and the cross roller bearing according to the present invention are installed and operated in harmonic reducers, respectively, and FIG. 7 is a graph illustrating stiffness variation with respect to moment before and after seal insertion in the conventional cross roller bearing and the cross roller bearing according to the present invention.

The cross roller bearing according to the present invention includes an outer ring 110 having an outer ring raceway 111 formed on an inner circumferential side thereof, and an inner ring 120 located inside the outer ring 110 and having an inner ring raceway 121 formed on an outer circumferential side thereof to face the outer ring raceway 111. A plurality of rolling elements 150 are provided between the outer ring raceway 111 and the inner ring raceway 121. The rolling elements 150 are arranged orthogonally to each other in the circumferential direction. A seal 180 is installed in an opening between the outer ring 110 and the inner ring 120.

In this instance, a seal insertion part 116 into which the seal 180 is inserted is formed in the outer ring 110. The seal insertion part 116 includes an insertion step portion 1161 extending outward in the axial direction, and a seal contact portion 1162 extending outward in the axial direction from the radial outer side of the insertion step portion 1161. In this instance, the seal insertion part 116 further includes an insertion guide portion 1163 formed on the axial outer side of the seal contact portion 1162 and an outer contact portion 1164 extending in the axial direction.

The inner ring raceway 121 and the outer ring raceway 111 have a V-shaped cross section, and the facing inner and outer ring raceways 121 and 111 form a square roller raceway. The adjacent rolling elements 150 are arranged orthogonally to each other and have a cylindrical shape.

The insertion guide portion 1163 is provided at an axial end of the outer contact portion 1164 in the form of a chamfer or a round, and serves to guide insertion of the seal 180.

The seal contact portion 1162 and the outer contact portion 1164 are formed in a cylindrical shape. In this instance, the inner diameter of the outer contact portion 1164 is larger than that of the seal contact portion 1162, which will be further described below.

The seal 180 includes a seal support 181 and a seal body 182 coupled to the seal support 181. The seal support 181 includes a support insertion portion 1811. The support insertion portion 1811 has a bent shape and extends inward in the axial direction from the radial outer side. The seal body 182 includes a body insertion portion 1821. The body insertion portion 1821 protrudes beyond the support insertion portion 1811 in the radial direction from the axial outer side of the support insertion portion 1811.

As the seal 180 is inserted into the seal insertion part 116, the support insertion portion 1811 comes into contact with the seal contact portion 1162, and the body insertion portion 1821 comes into contact with the outer contact portion 1164.

The seal support 181 may be made of steel, and the seal body 182 may be made of a material such as elastomer. The seal body 182 is coupled to the seal support 181.

The seal body 182 covers the axial outer side of an annular portion of the seal support 181 and also covers the inner end and a portion of the axial inner side of the annular portion of the seal support 181 in the radial inner direction.

The seal body 182 includes at least one seal lip 1822 protruding inward in the radial direction and is in contact with the outer circumferential surface of the inner ring.

A seal recess 183 is formed on the outer circumferential side of the seal 180. The seal recess 183 is concavely formed between the support insertion portion 1811 and the body insertion portion 1821.

A space S is formed among the seal recess 183, the seal contact portion 1162, a contact step portion 1165, and the outer contact portion 1164. In this instance, a portion of the body insertion portion 1821 moves into the space S while the body insertion portion 1821 deforms upon contact with the outer contact portion 1164.

In more detail, FIGS. 4 and 5 illustrate an overlapped state of the seal body 182 and the outer ring 110, indicating that deformation occurs in the portion where the body insertion portion 1821 overlaps with the outer ring 110.

As described above, the seal insertion part 116 includes the insertion step portion 1161 extending outward in the axial direction and the seal contact portion 1162 extending outward in the axial direction from the radial outer side of the insertion step portion 1161.

The seal insertion part 116 includes the contact step portion 1165 formed on the axial outer side of the seal contact portion 1162 and further includes the outer contact portion 1164 extending in the axial direction. Accordingly, when the seal 180 is inserted and assembled, deformation of the outer ring 110 by the seal 180 is reduced. In particular, the inner diameter of the outer contact portion 1164 is formed larger than that of the seal contact portion 1162. The support insertion portion 1811 comes into contact with the seal contact portion 1162, and the body insertion portion 1821 comes into contact with the outer contact portion 1164 to disperse pressure, thereby preventing deterioration of sealing performance and sharply reducing deformation of the outer ring 110 due to assembly of the seal 180.

In addition, when the seal 180 is assembled, the body insertion portion 1821 is deformed and moves into the space S, and a portion of the body insertion portion 1821 comes into contact with the seal contact portion 1162. However, it was confirmed that when, in the overlapped state before deformation of the seal body 182, the support insertion portion 1811 was brought into contact with the seal contact portion 1162 and the body insertion portion 1821 was brought into contact with the outer contact portion 1164, the above-mentioned effect occurred.

As described above, the seal recess 183 is formed on the outer circumferential side of the seal 180. The seal recess 183 is formed concavely between the support insertion portion 1811 and the body insertion portion 1821. Due to the seal recess 183, in the overlapped state before the seal 180 is inserted and deformed in the outer ring 110, the space S is formed among the seal recess 183, the seal contact portion 1162, the contact step portion 1165, and the outer contact portion 1164, so a portion of the seal body 182 moves into the space S due to deformation of the seal body 182.

Moreover, by increasing an overlap (D2-D1) between the body insertion portion 1821 and the outer ring 110, the sealing effect is maintained and deformation of the outer ring 110 caused by the seal body 182 is significantly reduced.

The body insertion portion 1821 includes inclined portions 18211-1 formed on both axial sides and has a shape in which the width in the radial direction decreases outward. In this case, an axial width L3 of a radial outer surface 18211 of the body insertion portion 1821 is formed to be smaller than the axial width L1 of the outer contact portion 1164. It should be noted that the above is described based on the state before deformation of the seal body 182.

In other words, in the overlapped shape before deformation of the seal body 182, the body insertion portion 1821 includes the inclined portions 18211-1 formed on both axial sides and has the shape in which the width in the radial direction decreases outward. In this case, the axial width L3 of the radial outer surface 18211 of the body insertion portion 1821 is formed to be smaller than the axial width L1 of the outer contact portion 1164. Accordingly, even if the overlap D2-D1 becomes large when the body insertion portion 1821 is deformed in contact with the outer contact portion 1164, deformation of the outer ring 110 is suppressed. In addition, the inclined portions on both sides reduce the resistance to deformation of the body insertion portion 1821, thereby preventing a localized large stress from being applied to the outer ring 110.

Meanwhile, the contact step portion 1165 has an inclination extending outward in the axial direction and inward in the radial direction. In this case, the bottom surface of the space S is formed by the seal body 182 and is spaced apart inward from the seal contact portion 1162 to face the seal contact portion 1162. In addition, among the inclined portions of the body insertion portion 1821 in the axial direction, a first inclined portion 18212 located on the axial inner side is spaced apart from the contact step portion 1165 and faces the contact step portion 1165.

According to the present invention, since the contact step portion 1165 is inclined, the body insertion portion 1821 is smoothly guided into the space S during deformation, thereby reducing resistance. As a result, the load applied to the outer ring 110 due to deformation of the body insertion portion 1821 is prevented from concentrating locally. Accordingly, excessive local deformation of the outer ring raceway 111 or the outer circumference of the outer ring 110 can be prevented, and deformation that may affect leakage or vibration can be suppressed overall.

Referring to FIG. 6, it can be seen that in the related art, as illustrated in FIG. 6(a), there is significant torque variation, resulting in a ripple phenomenon. However, according to the present invention, as illustrated in FIG. 6(b), torque variation is gentler, and the ripple phenomenon is reduced.

Furthermore, referring to FIG. 7, in the related art (see the left side of FIG. 7), the deformation amount before and after press-fitting increases as the moment increases. That is, angular deformation increases by 11 to 45% before and after press-fitting, indicating that raceway deformation occurs. In contrast, according to the present invention, as shown on the right side of FIG. 7, the angular deformation increases by only 4 to 8% before and after press-fitting, confirming that raceway deformation is improved.

According to the present invention, deformation of the outer diameter of the bearing and deformation of the raceway before and after seal assembly can be improved. Accordingly, rigidity against moment loads can be maintained, and torque ripple caused by excessive clearance in the raceway can be significantly reduced, thereby greatly reducing vibration when mounted on a robot or the like.

Meanwhile, when a radial distance measured from the seal contact portion 1162 to the outer surface 18211 of the body insertion portion is defined as D2, and a radial distance from the seal contact portion 1162 to the outer contact portion 1164 is defined as D1, a compression ratio (α) of the body insertion portion 1821 inserted into and compressed by the outer contact portion 1164 can be calculated as:

$$α = ( D 2 - D 1 ) / D 2.$$

Preferably, the compression ratio α is greater than 0 and less than 0.3.

It was confirmed that when α is 0.3 or more, the load applied to the outer ring 110 by the body insertion portion 1821 increased rapidly, resulting in a significant increase in deformation of the outer ring 110 and a sharp increase in lubricant leakage and vibration of the bearing. Additionally, a rapid decrease in moment stiffness of the bearing was confirmed.

Additionally, when an axial width of the outer contact portion 1164 is defined as L1, an axial width of the body insertion portion 1821 based on the seal contact portion 1162 is defined as L2, an axial width of the outer surface 18211 of the body insertion portion 1821 is defined as L3, and an inclination angle of the contact step portion 1165 with respect to the radial direction is defined as θ, a filling rate (β) of the seal body 182 can be calculated as:

$$β = [ ( L 2 \times D 2 ) - \{ ( L 2 - L 3 ) / 2 \times D 2 \} ] / [ ( L 1 \times D 1 ) + ( D 1 \times D 1 / 2 tan θ ) ] .$$

Preferably, the filling rate β is in the range of 0.6 to 0.9.

It is because when the filling rate β is less than 0.6, the sealing performance of the seal deteriorates, resulting in a sharp increase in lubricant leakage but, when the filling rate β is greater than 0.9, the load applied to the outer ring increases sharply, causing excessive deformation of the outer ring, a significant increase in lubricant leakage, and a rapid decrease in moment stiffness.

What is claimed is:

1. A cross roller bearing comprising:
an outer ring having an outer ring raceway formed on an inner circumferential side thereof;
an inner ring located inside the outer ring and having an inner ring raceway formed on an outer circumferential side thereof to face the outer ring raceway;
a plurality of rolling elements disposed between the outer ring raceway and the inner ring raceway, and orthogonally arranged in a circumferential direction; and
a seal installed in an opening between the outer ring and the inner ring,
wherein the outer ring includes a seal insertion part into which the seal is inserted, and the seal insertion part includes an insertion step portion protruding outward in an axial direction and a seal contact portion extending outward in the axial direction from a radial outer side of the insertion step portion, and
wherein the seal insertion part further includes
an insertion guide portion formed on an axial outer side of the seal contact portion and an outer contact portion extending in the axial direction.

2. The cross roller bearing according to claim 1, wherein the seal contact portion and the outer contact portion are formed in a cylindrical shape, and an inner diameter of the outer contact portion is greater than an inner diameter of the seal contact portion.

3. The cross roller bearing according to claim 1, wherein the seal includes a seal support and a seal body coupled to the seal support,
wherein the seal support includes a support insertion portion that is bent and extends axially inward from a radial outer side, and the seal body includes a body insertion portion protruding radially beyond the support insertion portion on an axial outer side of the support insertion portion, and
wherein the seal is inserted into the seal insertion part such that the support insertion portion comes into contact with the seal contact portion and the body insertion portion comes into contact with the outer contact portion.

4. The cross roller bearing according to claim 3, wherein a seal recess is concavely formed between the support insertion portion and the body insertion portion on an outer circumferential side of the seal, and
wherein a space is formed among the seal recess, the seal contact portion, a contact step portion, and the outer contact portion, and when the body insertion portion is deformed in contact with the outer contact portion, a portion of the body insertion portion moves into the space.

5. The cross roller bearing according to claim 4, wherein the body insertion portion includes inclined portions on both axial sides such that a width in a radial direction decreases outward, and an axial width of an outer surface of the body insertion portion is smaller than an axial width of the outer contact portion.

6. The cross roller bearing according to claim 5, wherein the contact step portion is inclined outward in the axial direction and inward in the radial direction, and
wherein a bottom surface of the space is formed by the seal body and inwardly spaced from the seal contact portion to face the seal contact portion, and a first inclined portion of the body insertion portion, which is disposed on an axial inner side of the body insertion portion, is spaced apart from the contact step portion to face the contact step portion.

7. The cross roller bearing according to claim 5, wherein when a radial distance measured from the seal contact portion to the outer surface of the body insertion portion is defined as D2, and a radial distance from the seal contact portion to the outer contact portion is defined as D1, a compression ratio α of the body insertion portion compressed by insertion into the outer contact portion is calculated as:
$$α = ( D 2 - D 1 ) / D 2.$$
and
wherein the compression ratio α is greater than 0 and less than 0.3.

8. The cross roller bearing according to claim 5, wherein when an axial width of the outer contact portion is defined as L1, an axial width of the body insertion portion based on the seal contact portion is defined as L2, an axial width of the outer surface of the body insertion portion is defined as L3, and an inclination angle of the contact step portion with respect to the radial direction is defined as θ, a filling rate (β) of the seal body is calculated as:
$$β = [ ( L 2 \times D 2 ) - \{ ( L 2 - L 3 ) / 2 \times D 2 \} ] / [ ( L 1 \times D 1 ) + ( D 1 \times D 1 / 2 tan θ ) ] .$$
and
wherein the filling rate β is in a range of 0.6 to 0.9.
